(19) United States
(12) Patent Application Publication
(10) Pub. No.: US 2024/0389855 A1
(43) Pub. Date: Nov. 28, 2024

(54) GUIDED SELF-CAPTURE OF DIAGNOSTIC IMAGES

(71) Applicant: Genentech, Inc., South San Francisco, CA (US)

(72) Inventor: George MAVROMARAS, South San Francisco, CA (US)

(21) Appl. No.: 18/793,540

(22) Filed: Aug. 2, 2024

Related U.S. Application Data
(63) Continuation of application No. PCT/US2023/012218, filed on Feb. 2, 2023.
(60) Provisional application No. 63/306,953, filed on Feb. 4, 2022.

(51) Int. Cl. A61B 3/14; G06V 40/18; G06V 40/20; G16H 30/20; H04N 23/63

(52) U.S. Cl. CPC A61B 3/14; G06V 40/193; G06V 40/20; G16H 30/20; H04N 23/631

(57) ABSTRACT

An app for guided self-capture of images may display a graphical user interface (GUI) on a screen of a mobile computing device with a camera. The GUI may present instructions to capture images of a specific area of concern on a patient's body, including a graphical representation of the specific area of the user's body posed in a predefined position. The GUI may display the graphical representation in conjunction with a view appearing in a lens of the camera. The app may then store one or more images captured by the camera and display, in the GUI, the one or more stored images together with instructions to review the stored images. In response to receiving user input approving one or more of the stored images, the app may then transmit the approved images to a system associated with a physician or clinician.

### CROSS-REFERENCE TO RELATED APPLICATION

This application is a continuation of International Application No. PCT/US2023/012218, filed on Feb. 2, 2023, which claims the benefit of U.S. Provisional Application No. 63/306,953 filed Feb. 4, 2022, the entire content of each of which is incorporated herein by reference in its entirety.

### TECHNICAL FIELD

This application relates generally to a user interface for an imaging application.

### BACKGROUND

Remote telehealth systems typically rely on a combination of videoconferences between patients and clinicians and/or physicians and submission of diagnostic information either captured by a monitoring device or captured by the patient themselves (e.g., taking a photo of an area of concern on the patient's body that can be visually assessed). For example, the patient may need to capture a photo of an area of the body where symptoms of a disease may be visible, an implant was inserted, surgery was previously performed, or treatment was administered. The problem with having the patient capture images themselves is that the patient may not be familiar with the type or quality of information required by a clinician and/or physician in order to competently assess the patient's condition.

For example, diagnostic photos captured by the patient may be captured from a viewpoint too distant to capture sufficient detail or too close to capture the entire area of interest. For example, the photos captured by the patient may show the patient's entire face, but fail to show sufficient detail around the eye. In another example, the photos captured by the patient that focus on a wound may not have included adjacent regions of the body that could have shown edema, redness, discoloration, or other signs of infection.

In addition, the patient's area of concern may be improperly posed, blurry or indistinct, or captured from a viewpoint or angle that does not meet a predefined standard. For example, the patient may need to capture a photo of an area of the body where symptoms of a disease may be visually presenting, an implant was inserted, surgery was previously performed, or treatment was administered. In such cases, the patient may need to move a limb into a particular position, pull aside a fold of skin, roll the eyes in a particular direction, or stretch a part of the body to provide better visibility of the area of concern. There is a need for a tool that can provide a patient with instructions and guidance for capturing usable diagnostic images of an area of interest on the patient's body for review (e.g., by a clinician or physician).

### SUMMARY

Example embodiments of a user interface of a software application (e.g., a mobile device app executing on a mobile device including a camera) as described herein may provide instructions to a user of the mobile device to take photos capturing usable diagnostic images of an area of concern of the user's body. The user interface of the mobile device app may provide textual, visual and/or audio instructions and/or feedback regarding how to position the camera lens and/or how a user of the mobile device should pose their body to capture usable diagnostic images of the area of concern of their body. The instructions regarding how to pose the user's body may include a graphical representation showing how to pose the user's body to capture a usable diagnostic image of the area of concern of the user's body. The graphical representation may comprise one or more static images or an animation. The graphical representation may be displayed together with an image of the view appearing through the lens of the camera.

In particular embodiments, the mobile device app may analyze the image captured by the camera of the mobile device (prior to taking the photo, while taking the photo, or after the photo has been taken) to ensure that the image complies with a profile of a predefined position. Analysis may be performed using techniques involving image analysis to detect and identify relevant features, then measuring attributes of any identified relevant features. The measured attributes may then be assessed in light of the profile of the predefined position.

The user interface may also provide feedback to the user if the camera position and/or body pose needs to be adjusted. Once the camera is in position and the user's body is posed, the user interface may enable the user to indicate that they are ready for a photo to be taken (e.g., by tapping an interactive element, such as a button, displayed in the user interface, by speaking a voice command, or by making a gesture), at which point one or more photos may be taken. Alternatively, the mobile device app may analyze the image, detect the moment when the image meets one or more factors for determining that the image is within the range for a usable diagnostic depiction of the area of concern, and automatically start taking photos of the image being captured.

Photos may be taken using a “burst mode” that captures several photos in rapid succession within an extremely short period of time (e.g., 10 photos per second) in order to account for any movement while the photos are being captured. The user interface may provide the user with the option to retake a photo, and in some embodiments, the user interface may analyze the image(s) captured in the photo(s) and provide the user with a recommendation regarding whether or not the photos should be retaken. Such analysis may include, for example, analyzing photographic properties of the image, the properties including brightness, sharpness, contrast, etc. in comparison to predefined standards for the photographic properties.

The user interface may provide an option for the user to send the photo(s) to an image analyst (e.g., a physician or clinician). The user interface may provide the user with the ability to select one recipient from a list of image analysts to which their photos can be submitted, as well as the ability to review photos previously sent to a particular image analyst. The user interface may also provide alerts or notifications when the patient needs to take updated photos and/or submit new photos (e.g., on a periodic basis, in order to monitor progress).

In some embodiments, the mobile device app may be configured for use by an image analyst (e.g., a physician or clinician) to receive, review, annotate, classify, store, and/or request resubmission of photos by subject users (e.g., patients). Such embodiments of the mobile device app may also include the image capture workflow, so that the mobile device can be used in self-capture mode by a subject user, as described above (e.g., a nurse at a physician's office may hand the mobile device to a patient and teach them how take photos of their area of concern). The user interface of the mobile device app may provide access to all photos submitted by a subject user, not just the most recently submitted photos. If the user interface provides access to photos of more than one subject user, an image analyst may have the option to review photos of one subject user at a time, or to review and/or compare photos from more than one user at a time. The user interface may also provide the option for the image analyst to request that the submitting subject user retake a photo. When reviewing one or more photos, physicians may have the option to add annotations to one or more of the photos, which may be saved and retrieved later in conjunction with the photo.

### BRIEF DESCRIPTION OF THE DRAWINGS

FIG. 1 illustrates an overview of an example system for guided self-capture of diagnostic photos.

FIG. 2 illustrates a flowchart illustrating steps of a method for providing a user interface for guided self-capture of diagnostic photos.

FIG. 3 illustrates an example user interface displaying a graphical representation of an eye posed in a predefined position depicted together with an image of a user's eye as captured by a camera.

FIG. 4 illustrates an example user interface displaying a status message and progress indicator during the process of taking a photo.

FIG. 5 illustrates an example user interface for reviewing a photo taken by the user with an option to retake the photo if needed and an option to submit the photo.

FIG. 6 illustrates an example computer system.

### DESCRIPTION OF EXAMPLE EMBODIMENTS

Example embodiments of a user interface of a software application (e.g., a mobile device app executing on a mobile device including a camera) as described herein may provide instructions to a user of the mobile device regarding how to capture usable diagnostic images of an area of concern of the user's body. The user interface of the mobile device app may provide textual, visual and/or audio instructions and/or feedback regarding how to position the camera lens and/or how a user of the mobile device should pose their body to capture usable diagnostic images of the area of concern of their body. The instructions may direct the user to position the camera lens at a particular distance, height, or angle with respect to the area of concern. The instructions may direct the user to utilize one or more features of software associated with the camera in order to zoom in on the area of concern, identify a specific feature on which to auto-focus, enable or disable flash, increase or decrease exposure time, change aspect ratio, or set a timer to delay taking a photo. The instructions may direct the user to pose and/or manipulate (e.g., with the user's own hand or foot, or with the assistance of another person) a part of the body to reveal, isolate, emphasize, highlight, or otherwise provide greater visibility of the body part.

The instructions regarding how to pose the user's body may include a graphical representation showing how to pose the user's body to capture a usable diagnostic image of the area of concern of the user's body. The graphical representation may comprise one or more static images or an animation. The graphical representation may comprise a stylized depiction of the area of concern in the desired pose. The graphical representation may be displayed together with an image of the view appearing in the lens of the camera (e.g., the patient's face as captured by a user-facing camera of the mobile device).

A set of instructions regarding how to position the camera and how to pose the user's body may be displayed when the app is first opened. The instructions may be text, other visual content, audio content, or some combination thereof. The instructions may be presented prior to and/or during the image capture process. By presenting the instructions in a step-by-step manner during the image capture process, the user interface may enable the user to perform each instruction as it appears. The app may display one instruction at a time, and only display the next instruction after confirming that the current instruction has been satisfied.

The user interface may also provide feedback to the user if the camera position and/or body pose needs to be adjusted. Once the camera is in position and the user's body is posed, the user interface may enable the user to indicate that they are ready for a photo to be taken (e.g., by tapping an interactive element, such as a button, displayed in the user interface, by speaking a voice command, or by making a gesture), at which point one or more photos may be taken. Alternatively, the mobile device app may analyze images of the view appearing in the lens of the camera to assess compliance with a predefined standard for a usable diagnostic image of the specific area of concern. As the user poses their body to adjust their position, the mobile device app may detect the moment when the images comply with the predefined standard for a usable diagnostic image of the area of concern, and automatically start taking photos at that moment.

The predefined standard for a usable diagnostic image may comprise predefined standards for photographic properties of an image, a profile of a predefined position as depicted in a usable diagnostic image. or other requirements to achieve a usable diagnostic image. Photographic properties of the image may include, for example, brightness, sharpness, contrast, focus, framing, etc. The profile of the predefined position may specify a minimum threshold, maximum threshold, ratio-based threshold, acceptable range, acceptable type, acceptable degree of deviation, or any other appropriate metric for measurable attributes of relevant features of the image. Analysis may be performed using techniques involving image analysis to detect and identify relevant features and may involve measuring attributes of any identified relevant features. Measurable attributes may comprise: visibility of one of the identified features, a color of one of the identified features, an area of one of the identified features, a distance between two of the identified features, a proportion of one of the identified features to another one of the identified features, a shape of one of the identified features, a position of one of the identified features, a location of one of the identified features, an orientation of one of the identified features, a quantity of the identified features of a particular type, an angle associated with one or more identified features, or a ratio of measurable attributes.

Photos may be taken using a “burst mode” that captures several photos in succession within an extremely short period of time (e.g., 10 photos per second) in order to account for any movement while the photos are being captured. The user interface may provide the user with the option to retake any of the captured photos. In some embodiments, the app may analyze the captured image(s) to assess whether the images comply with the predefined standard for a usable diagnostic image of the area of concern and provide the user with a recommendation regarding whether or not the captured images should be retaken.

The user interface may provide an option for the user to send the photo(s) to an image analyst (e.g., a physician or clinician). The user interface may provide the user with the ability to select one recipient from a list of image analysts to which their photos can be submitted, as well as the ability to review photos previously sent to a particular image analyst. The user interface may also provide alerts or notifications when the patient needs to take updated photos and/or submit new photos (e.g., on a periodic basis, in order to monitor progress).

In some embodiments, the mobile device app may be configured for use by an image analyst (e.g., a physician or clinician) to receive, review, annotate, classify, store, and/or request resubmission of photos by subject users (e.g., patients). Such embodiments of the mobile device app may also include the image capture workflow, so that the mobile device can be used in a self-capture mode by a subject user, as described above (e.g., a nurse at a physician's office may hand the mobile device to a patient and teach them how take photos of their area of concern). The user interface of the mobile device app may provide access to all photos submitted by a subject user, not just the most recently submitted photos. If the user interface provides access to photos of more than one subject user, an image analyst may have the option to review photos of one subject user at a time, or to review and/or compare photos from more than one user at a time. The user interface may also provide the option for the image analyst to request that the submitting subject user retake a photo. When reviewing one or more photos, physicians may have the option to add annotations to one or more of the photos, which may be saved and retrieved later in conjunction with the photo.

The aforementioned elements may be exemplified by applying them to an example case of capturing photos of the eye. The relevant set of instructions may include 1) a reminder to ensure that image capture lighting meets predetermined standards or thresholds, 2) step-by-step instructions illustrating or directing how the patient should lift their upper eyelid and then move their pupil downwards, 3) a graphical representation of an eye posed in a predefined position for medical assessment, (4) instructions to capture an image, and 5) any post-photo-capture steps. The instructions may be displayed over a series of screens, with at least one instruction per page, and each instruction may comprise a visual and/or a text component, as well as annotations or visual, auditory, or tactile cues. Displaying the instructions as part of the photo capture process may be done by first asking the patient to find a spot with good lighting, and then waiting until sufficient lighting is detected before presenting the next instruction, namely the graphical representation of the eye posed in the predefined position and how the patient should pose their eye by pulling up the upper eyelid and looking downward and towards their nose. Once the app either detects that the patient has taken a photo (or automatically taken a photo upon detecting that the user posed their eye correctly), the next instruction may be displayed, and the process repeats with the final step. If displaying both upfront instructions and shortened key components, the upfront instructions may be done in the same manner as described earlier, while key components like how to lift the upper eyelid and how to use the visual guide may be overlaid on the camera. The overlay may then be easily removed with some simple action, such as touching the screen.

In particular embodiments. the mobile device app may analyze the visual information from the camera (prior to capture or during capture) to ensure that the photo(s) captured will meet certain requirements and provide feedback if the camera position and/or body pose needs to be adjusted. The app may include a feature that provides real-time feedback on different aspects of the photo capturing process before and perhaps even while the photos are being taken. Such feedback may include notifying the patient when they are too close to the camera, when they are too far, when they have not aligned the body part closely enough with the visual guide, when the body part is not properly posed, when something is obstructing the body part, when the camera has gone out of focus, when the lighting has become insufficient, when the camera may be placed at a poorly positioned viewpoint, and many others. The feedback may include a description of the problem and how the patient can resolve the problem. Generating the feedback may be achieved by any means, such as with an artificial intelligence, statistical, or machine learning computational model that is trained to detect when one of the aforementioned problems arises.

The computational model may be one that extends the artificial intelligence model used for instructions that was discussed earlier or a completely separate model. Different feedback may also be generated with different means. The manner in which the feedback is made available to the patient may also be done through numerous methods. One approach may be to display a notification message somewhere on the screen with the associated feedback. However, because the patient may not always be able to readily notice purely visual notifications, such as in the example case where photos of the eye are being captured which may require the patient's pupil to be directed away from the device screen, the app could also incorporate some kind of haptic notification to alert the patient. The app may also provide multiple forms of notification, such as by combining visual and haptic notifications.

Once the camera is in position and the patient's body is posed, any number of photos may be taken, with the option to retake as many times as needed. The app may include a number of “modes” through which to capture photos. One of the modes may be a “burst mode” that captures several photos in succession within an extremely short period of time (e.g., 10 photos per second) in order to mitigate the effect of movement while the photos are being captured. This mode may be designed so that as long as the camera button is pressed, the app will continue taking photos, with the total number of photos captured proportional to the length of time that the button was pressed. A similar mode to that of the burst mode may be one that also continuously captures photos in quick succession, but instead of requiring that the button be pressed the entire time, a first tap starts the continuous capture process, with the process ending either with a second tap of the button (“double touch mode”) or after a predetermined time frame (“timed mode”). With these modes, the app may allow various levels of customizability, such as allowing the patient to change the number of photos captured per second or how long the predetermined capture time will be. Another “countdown mode” may introduce a countdown when the button is first pressed, giving the patient some extra time to perfect their positioning before photos are captured.

These modes do not preclude the app also offering a simple “single photo mode” where only a single photo is captured on the release of the button, regardless of how long the button had been pressed before it was released. The app may allow multiple modes to be active at once, as long as the modes do not innately oppose one another, so the burst mode may be coupled with the countdown mode, but the burst mode may not be able to work with the single photo mode. For modes that may involve some kind of timed component, such as a timed or countdown mode, the app may provide some kind of notification when the relevant time period ends, since the patient may not be looking at the screen and thus may not know when the process has finished. By notifying the patient, the app may help prevent them from moving before the timed process ends (such as if the patient moves too early when trying to check the status of the capture process), but it may also help to prevent them from having to hold the position for too long after the timed process ends.

To try to ensure that the quality of the photos captured does not degrade suddenly in the middle of the capturing process, particularly in modes that may capture photos in rapid succession for an amount of time, the app may be automatically terminate the capturing process when it detects a substantial change in the visual quality. Changes that may trigger this early termination may be anything that could result in unusable diagnostic photos and may be similar to the causes that the app may detect prior to beginning to capture photos, such as if the patient moves too far away from the camera, if the patient is no longer aligned closely enough to the visual guide, or the camera suddenly goes out of focus. If this early termination is triggered, the app may save whatever had been captured up until that point or simply discard everything and require starting a brand new capture process from the beginning. The app may allow this feature to be customized by the user, perhaps including the option to disable the feature completely or adjust the termination's trigger threshold.

The photo capture process and the associated set of features may also be extended to support taking photos of multiple body parts. For such embodiments, once the process is complete for one body part, there may be an option to buffer the photos that were just taken while completing the process again with another body part. When the patient is satisfied with all the photos from all the body parts they wish to submit, they may complete the photo capture process and proceed to the next step.

In particular embodiments, the app may have a post-photo-capture step reserved to review the photos that were just captured. The app itself may first analyze the photos after they are captured to perform a preliminary review of the photos. This may be done in any number of ways, such as using the same artificial intelligence model that analyzed visual information before and during the photo capture process. Any photos that the app's artificial intelligence model detects as potentially unsatisfactory (e.g. the patient was not posed adequately, there was some kind of obstruction, the photo turned out blurry, etc.) may be marked in some way, such as with an exclamation, to reflect that fact before being presented to the patient along with the other captured photos. The patient may manually review the photos they took.

During the review process, the app may include various features to aid the patient in their review. The app may display an example photo of the same body part that was just captured along with the visual guide that was just used overlaid on top of the photo so that the patient may use the example photo as a standard against which to evaluate their own photos. If the patient is satisfied with the photo, they may approve the photo for submission; if the patient is not satisfied with the photo, they may discard the photo. If the patient took multiple photos and thus is now reviewing multiple photos, the app may either display one photo at a time and have the patient review all the photos one by one, or display multiple photos at a time so the patient may review multiple photos at a time. For the latter, the app may need to ensure that each of the displayed photos are large enough for the patient to adequately review them, which may limit the number of photos that can be displayed at any given time. The app may also take an approach where multiple photos are displayed, but individual photos may then be selected and enlarged for more detailed review. This review step may be scheduled to happen either after all photos have been captured or after each photo or set of photos if using a mode such as the burst mode.

If the patient captured a video of the body part, the visual guide may be overlaid on each video for its entire duration to help the patient determine if they may have moved too far away from the indicated position at any point during the video. Otherwise, the review process for videos may be largely similar to that of photos. Since the patient may have taken both photos and videos, the app may allow the patient to review photos and videos at the same time, or may separate photos and videos into separate review sessions.

In particular embodiments, the mobile device app may be configured for a patient sending photos to a physician or clinician. In addition to the user interface of the mobile app that provides guidance regarding how to position the camera lens and/or how a patient's body should be posed, the mobile device app may provide the patient with the ability to select which physician and/or clinician to submit their captured photos to, and to review previously captured and/or previously submitted photos, as well as providing alerts or notifications when the patient needs to take replacement photos and/or submit new photos (e.g., on a periodic basis, in order to monitor progress). In regards to submitting captured photos, after the patient has reviewed the photos they captured and selected the physician(s) and/or clinician(s) they wish to send the photos to, the app may either submit the photos immediately or first buffer the photos locally until the patient has finished taking photos of any other body parts they wish or need to take before submitting all the photos in bulk. If the app buffers the images, the app may remind the patient, perhaps intermittently, with some notification that they still need to confirm the submission of the photos that were captured. The reminder may be done in any manner, whether as a banner, as a pop up, or anything else. The app may allow the patient to freely switch between having the app submit photos immediately or buffer and submit in bulk.

In particular embodiments, the mobile device app may be configured for a physician or clinician who may need to receive, review, annotate, classify, store, and/or request resubmission of patient photos, as well as handing their mobile device to patients to capture photos as described above, or capturing photos of a patient themselves. The app may alert the physicians or clinicians with a notification as soon as new patient photos are submitted. Physicians or clinicians may have access to all photos associated with a patient, not just the most recently submitted photos. Physicians or clinicians may also have access to photos for multiple patients or even for all the patients that the app has data on. While the physician/clinician access to photos may be completely unrestricted, the app may offer the option to implement various administrative restrictions, such as only allowing physicians to view the photos of patients for whom they are personally responsible.

When physicians review photos, they may review the photos of one particular patient at a time, or review photos from more than one patient at a time. The app may allow physicians to add any amount of annotations for each photo, which may then be saved and retrieved later in conjunction with the photo. The app may provide a number of tools to help the physicians in their review process. For example, the app may enable physicians to customize annotations to more easily identify their comments later on. Annotations may be color coded to represent different kinds of developments associated with a photo, e.g. red for a photo that may reveal very concerning developments which require the patient to schedule a clinic visit, yellow for a photo that may suggest something that might become concerning later on, and green for a photo that may suggest everything is fine with the patient. Other tools and features may include built-in markings to reflect different developments, such as a star to represent photos of particularly good quality, check marks to represent good or safe developments in the patient's condition, or a house to represent a need for the patient to schedule a clinic visit.

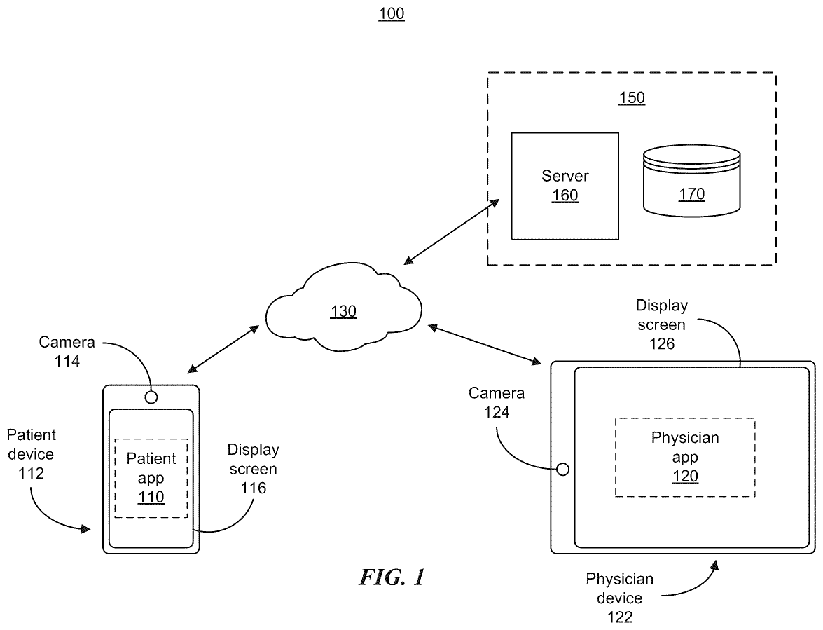

FIG. 1 illustrates an example system 100 for guided self-capture of diagnostic photos. Mobile device application (e.g., patient app 110) executes on a mobile computing device (e.g., patient device 112) that supports the app. Camera 114 may be built in to patient device 112. If patient device 112 only has a single camera, whether it be a front-facing camera 114 positioned above a display screen 116 of patient device 112 or a rear-facing camera positioned on an opposite side of the display screen 116 of patient device 112, that will be the one used to take the photos. If patient device 112 has both a front-facing and rear-facing camera, patient app 110 may use either camera to take the photos. Patient app 110 may be able to connect with a third-party camera controlled by patient device 112. Patient app 110 connect with a network 130 in order to send the patient data to clinical data system 150. The user interface for the app may also be done in any number of ways, with examples of certain approaches discussed later in the section.

Physicians may interact with a mobile application 130 (e.g., physician app 120), which may similarly execute on a mobile device (e.g., physician device 122) that supports physician app 120. The physician app 120 may connect with network 130 in order to retrieve patient data and photos from clinical data system 150 and submit and save any annotations or comments. The same communication protocol used to establish the connection between the app 110 and the network 130 may be used to establish the connection between the physician app 130 and the network 130. The user interface of physician app 120 may enable reviewing, annotating, classifying, ranking, scoring, storing, and/or requesting resubmission of patient photos.

Physician app 120 may also provide the ability to capture photos using camera 124 of physician device 122 (shown in FIG. 1 as a front-facing camera positioned adjacent to display screen 126). For example, if a patient checks in with a clinician at a clinical facility to check on the status of an area of concern, as part of the visit, the clinician's assistant may hand physician device 122 to the patient so that the patient can take photos of the area of concern while they are waiting for the clinician. Such photos may be stored as part of the data collected during the visit. In addition, if the patient needs assistance learning how to take photos on their own, the clinician may provide guidance during the visit by having the patient try capturing photos of themselves using physician device 122.

Clinical data system 150 may comprise a server 160 that responds to requests from patient app 110 and physician app 120 and a data storage 170 that handles securely and robustly storing the clinical data. Server 160 may check, clean, format, and store new patient photos as well as physician annotations and comments on the patient photos. Server 160 may clean the patient photos to remove any photo metadata that is not needed, confirm that the received photos meet or pass predetermined quality standards, and/or verify that the received photos capture diagnostic features of the patient. The server may, if necessitated by the type and design of the data storage, perform operations on the photo so it may be effectively stored into the data storage. The server may store the photo in data storage 170. This specific server design and associated methodology is solely exemplary, and particular embodiments may reorder, add, or remove steps to fit their specific needs.

When receiving a request from physician app 120 to review patient photos, the server 160 may check that the physician is allowed to access the patient's data, sending a rejection response immediately if they do not have access. Otherwise, the server may retrieve the data from storage, reformat the data if necessary from how they are stored to one that is human-readable, add any necessary metadata, and send the photos to physician app 120.

Server 160 may include several additional features for security and robustness purposes. For example, server 160 may encrypt data when writing to the data storage and decrypt any data when reading from the storage. The encryption method used may be any that is known in the art, such as Advanced Encryption Standard (AES) or Rivest-Shamir-Adleman (RSA). All such designs for the server are purely exemplary, and particular embodiments may adjust the process as necessary to fit specific needs. Finally, a dedicated data storage component 170 may house all of the clinical data, including photos, annotations and comments, metadata, and other relevant data.

In particular embodiments, clinical data system 150 may be capable of providing a selection of types of instructions for guided self-capture of different areas of concern. A physician or clinician may be able to select one of the types of instructions for a particular patient. The patient app may connect with clinical data system 150 in order to determine and/or download the specific instructions, including the specific graphical representation, to display for the patient. In some embodiments, the types of instructions may differ not only by the area of concern to be captured, but also by the specific symptom, indication, or other concern that the physician or clinician wants to focus on.

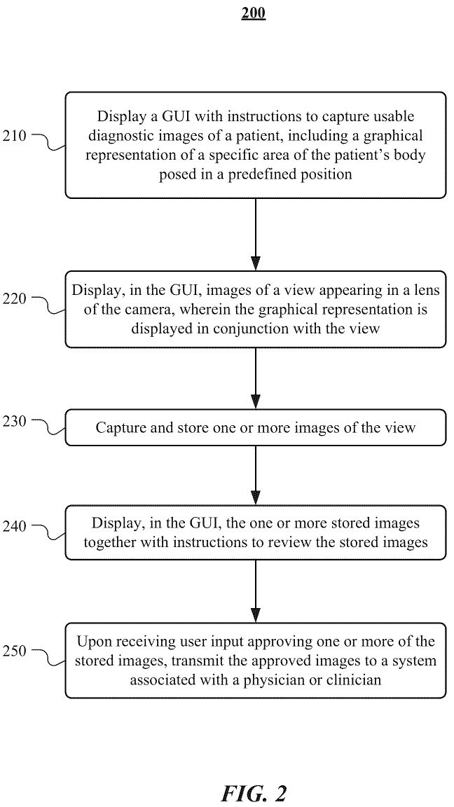

FIG. 2 illustrates an example method 200 for guided self-capture of diagnostic photos. The method may begin at step 210, where a mobile device application executing on a mobile device may display a graphical user interface (GUI) providing instructions to a patient regarding self-guided capture of usable diagnostic images of the patient. The GUI may include a graphical representation depicting a specific area of the patient's body posed in a predefined position. As described further herein, the instructions may be presented in a number of different ways. At step 220, the mobile device application may display images of a view that appears in a lens of the camera with the graphical representation of how to pose the body part displayed in conjunction with the view from the camera lens. As described further herein, the graphical representation may be displayed in a number of different ways, and may operate together with the instructions to guide the user on how to pose the body part. At step 230, the mobile device application may capture one or more images of the view that appears in the lens of the camera and store the captured images in local data storage on the mobile computing device. As discussed further herein, the images may be captured through a number of different “modes”. At step 240, the stored images may be displayed in the GUI with various instructions to review the images. As described further herein, various other elements may be displayed on the GUI at this time to help the user to review the images. At step 250, after receiving user input approving one or more of the stored images, the approved images may be transmitted to a system associated with a physician or clinician for their review and/or annotation.

Particular embodiments may repeat one or more steps of the method of FIG. 2, where appropriate. Although this disclosure describes and illustrates particular steps of the method of FIG. 2 as occurring in a particular order, this disclosure contemplates any suitable steps of the method of FIG. 2 occurring in any suitable order. Moreover, although this disclosure describes and illustrates an example method for guided self-capture of diagnostic photos including the particular steps of the method of FIG. 2, this disclosure contemplates any suitable method for guided self-capture of diagnostic photos including any suitable steps, which may include all, some, or none of the steps of the method of FIG. 2, where appropriate. Furthermore, although this disclosure describes and illustrates particular components, devices, or systems carrying out particular steps of the method of FIG. 2, this disclosure contemplates any suitable combination of any suitable components, devices, or systems carrying out any suitable steps of the method of FIG. 2.

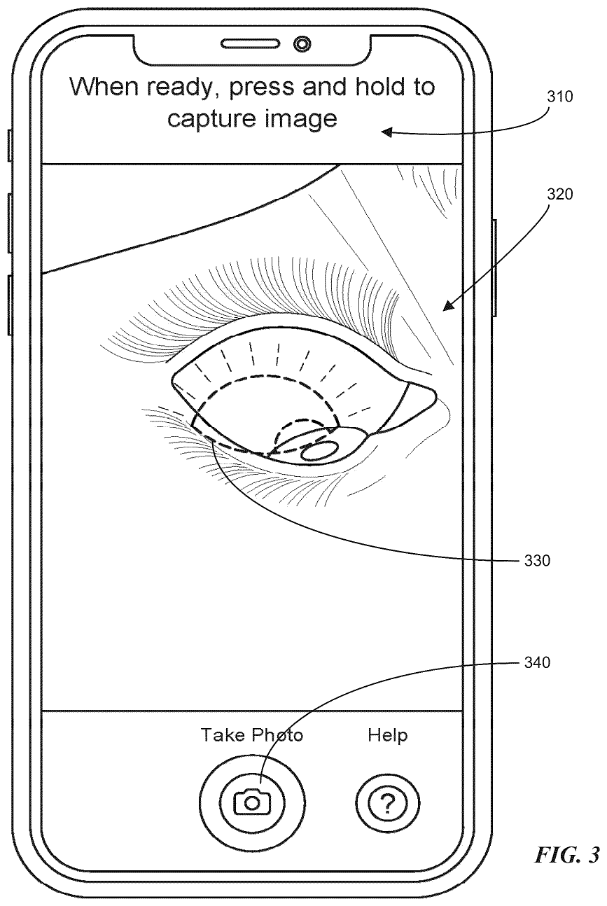

FIG. 3 illustrates an example of what the application may show during the pre-photo-capture as it is guiding the patient towards the predefined position. In this case, the patient is using the patient app to capture photo(s) of the eye using camera 114, with the eye posed to reveal an area of the sclera where an implanted device has been inserted. The message area 310 at the top of the screen may present instructions directing the patient to pose the eye by using a finger to manipulate the skin of the upper eyelid while rolling the eye downward and toward the nose, followed by instructions to capture the photo when they are ready. As illustrated, key aspects of the instructions may be reiterated at the time the patient prepares to pose, after the initial display of the detailed instructions. Display area 320 may show the image being captured by camera 114, together with an overlay of graphic representation 330. Graphic representation 330, which takes the form of a stylized eye, reflects a pose where a wide-open eye is rolled downwards and inward toward the nose (i.e., toward a medial canthus of the eye) so as to reveal both a large area of the sclera between an upper eyelid of the eye and an iris of the eye and a large area of the sclera between the iris and a lateral canthus of the eye. The camera button 340 at the bottom with the “Take Photo” text may suggest the current mode may take a single photo or a burst of photos upon the release of the button, based on how patient app 112 is configured.

As shown in FIG. 3, graphic representation 330 is shown as a line drawing of a stylized eye in the desired pose, using dashed lines; however, in particular embodiments, graphic representation 330 may be depicted as an outline, a transparent image, or an animation.

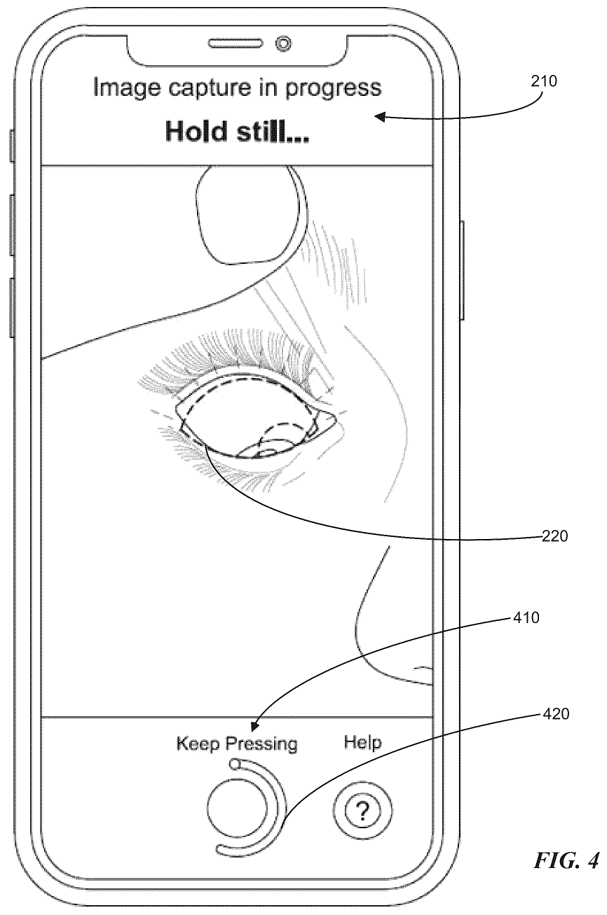

FIG. 4 illustrates what the user interface of the patient app may look like as it is actively photographing the patient's eye. As the photo capturing process continues, the patient should maintain the same pose, pulling back their upper eyelid and keeping their eye rolled downward and inward while also holding the camera in a fixed position laterally and at a fixed distance, so as to ensure persistent alignment with graphic representation 320. Based on the text in message area 310 at the top of the screen and text 410 above the photo capture button at the bottom, the current photo mode may be in the “burst mode” where multiple photos are being continuously taken as long as the button is pressed. The button text instructing the patient to continue pressing the button may thus be to ensure a complete burst of photos is captured. In some embodiments, a progress indicator 420 may be displayed while the burst of photos is being captured and stored, in order to let the user know when the process is complete. Similar embodiments may be used to capture a short video clip.

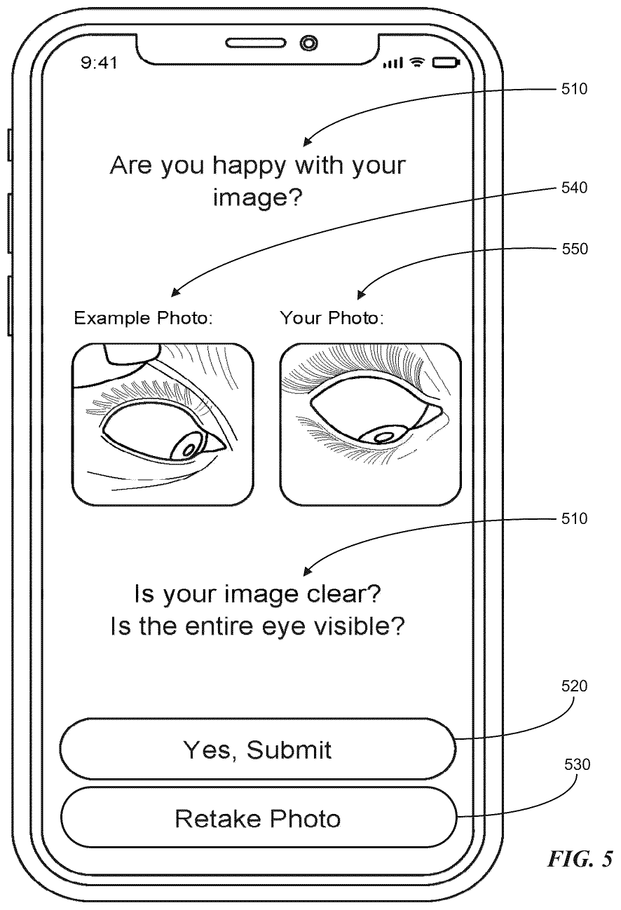

FIG. 5 illustrates an example of the user interface of the patient app at the review stage, when patients review the photos they captured. At the review stage, the patient is asked to consider several questions 510 prior to determining whether to click button 520 to accept and submit the photo or button 530 to retake the photo. In this example, the app displays an example photo 540 of a patient's eye where they pull the upper eyelid while moving their pupil downwards. The photo 550 that the patient captured is placed next to example photo 540, giving the patient an easy reference with which to evaluate their own photo. Questions 510 may provide further guidance on how the patient should evaluate the photo they captured. Although only one captured photo is shown in the example, this does not necessarily mean that the patient only captured one photo—the patient may have captured multiple photos, just that the app is only displaying one photo at a time for the patient to review. After reviewing their photo, the patient has the option to either submit the photo if they are satisfied with how the photo turned out or retake the photo if they are not satisfied. Upon clicking button 520, the patient app may upload photo 550 immediately upon clicking or wait until all photos have been reviewed before submitting in bulk. Likewise for the retake option, it could either discard the photo before retaking a new photo or hold the original photo so the patient may perhaps compare the new and original photos before returning to the review page with the better of the two.

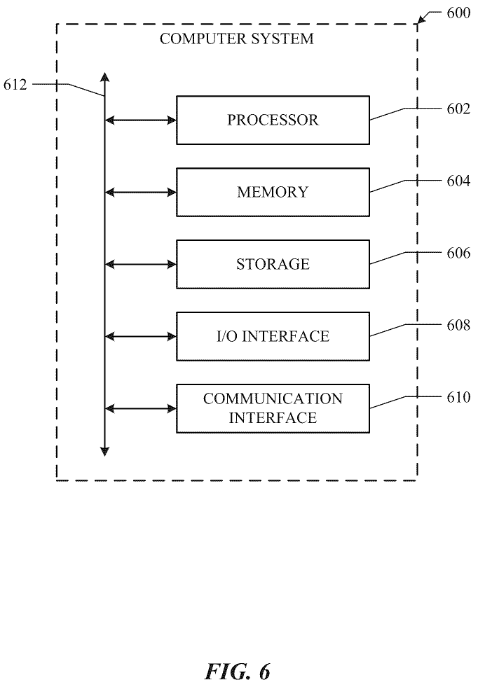

FIG. 6 illustrates an example computer system 600. In particular embodiments, one or more computer systems 600 perform one or more steps of one or more methods described or illustrated herein. In particular embodiments, one or more computer systems 600 provide functionality described or illustrated herein. In particular embodiments, software running on one or more computer systems 600 performs one or more steps of one or more methods described or illustrated herein or provides functionality described or illustrated herein. Particular embodiments include one or more portions of one or more computer systems 600. Herein, reference to a computer system may encompass a computing device, and vice versa, where appropriate. Moreover, reference to a computer system may encompass one or more computer systems, where appropriate.

This disclosure contemplates any suitable number of computer systems 600. This disclosure contemplates computer system 600 taking any suitable physical form. As example and not by way of limitation, computer system 600 may be an embedded computer system, a system-on-chip (SOC), a single-board computer system (SBC)(such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 600 may include one or more computer systems 600; be unitary or distributed; span multiple locations; span multiple machines; span multiple data centers; or reside in a cloud, which may include one or more cloud components in one or more networks. Where appropriate, one or more computer systems 600 may perform without substantial spatial or temporal limitation one or more steps of one or more methods described or illustrated herein. As an example and not by way of limitation, one or more computer systems 600 may perform in real time or in batch mode one or more steps of one or more methods described or illustrated herein. One or more computer systems 600 may perform at different times or at different locations one or more steps of one or more methods described or illustrated herein, where appropriate.

In particular embodiments, computer system 600 includes a processor 602, memory 604, storage 606, an input/output (I/O) interface 608, a communication interface 610, and a bus 612. Although this disclosure describes and illustrates a particular computer system having a particular number of particular components in a particular arrangement, this disclosure contemplates any suitable computer system having any suitable number of any suitable components in any suitable arrangement.

In particular embodiments, processor 602 includes hardware for executing instructions, such as those making up a computer program. As an example and not by way of limitation, to execute instructions, processor 602 may retrieve (or fetch) the instructions from an internal register, an internal cache, memory 604, or storage 606; decode and execute them; and then write one or more results to an internal register, an internal cache, memory 604, or storage 606. In particular embodiments, processor 602 may include one or more internal caches for data, instructions, or addresses. This disclosure contemplates processor 602 including any suitable number of any suitable internal caches, where appropriate. As an example and not by way of limitation, processor 602 may include one or more instruction caches, one or more data caches, and one or more translation lookaside buffers (TLBs). Instructions in the instruction caches may be copies of instructions in memory 604 or storage 606, and the instruction caches may speed up retrieval of those instructions by processor 602. Data in the data caches may be copies of data in memory 604 or storage 606 for instructions executing at processor 602 to operate on; the results of previous instructions executed at processor 602 for access by subsequent instructions executing at processor 602 or for writing to memory 604 or storage 606; or other suitable data. The data caches may speed up read or write operations by processor 602. The TLBs may speed up virtual-address translation for processor 602. In particular embodiments, processor 602 may include one or more internal registers for data, instructions, or addresses. This disclosure contemplates processor 602 including any suitable number of any suitable internal registers, where appropriate. Where appropriate, processor 602 may include one or more arithmetic logic units (ALUs); be a multi-core processor; or include one or more processors 602. Although this disclosure describes and illustrates a particular processor, this disclosure contemplates any suitable processor.

In particular embodiments, memory 604 includes main memory for storing instructions for processor 602 to execute or data for processor 602 to operate on. As an example and not by way of limitation. computer system 600 may load instructions from storage 606 or another source (such as, for example, another computer system 600) to memory 604. Processor 602 may then load the instructions from memory 604 to an internal register or internal cache. To execute the instructions, processor 602 may retrieve the instructions from the internal register or internal cache and decode them. During or after execution of the instructions, processor 602 may write one or more results (which may be intermediate or final results) to the internal register or internal cache. Processor 602 may then write one or more of those results to memory 604. In particular embodiments, processor 602 executes only instructions in one or more internal registers or internal caches or in memory 604 (as opposed to storage 606 or elsewhere) and operates only on data in one or more internal registers or internal caches or in memory 604 (as opposed to storage 606 or elsewhere). One or more memory buses (which may each include an address bus and a data bus) may couple processor 602 to memory 604. Bus 612 may include one or more memory buses, as described below. In particular embodiments, one or more memory management units (MMUs) reside between processor 602 and memory 604 and facilitate accesses to memory 604 requested by processor 602. In particular embodiments, memory 604 includes random access memory (RAM). This RAM may be volatile memory, if appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 604 may include one or more memories 604, where appropriate. Although this disclosure describes and illustrates particular memory, this disclosure contemplates any suitable memory.

In particular embodiments, storage 606 includes mass storage for data or instructions. As an example and not by way of limitation, storage 606 may include a hard disk drive (HDD), a floppy disk drive, flash memory, an optical disc, a magneto-optical disc, magnetic tape, or a Universal Serial Bus (USB) drive or a combination of two or more of these. Storage 606 may include removable or non-removable (or fixed) media, where appropriate. Storage 606 may be internal or external to computer system 600, where appropriate. In particular embodiments, storage 606 is non-volatile. solid-state memory. In particular embodiments, storage 606 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 606 taking any suitable physical form. Storage 606 may include one or more storage control units facilitating communication between processor 602 and storage 606, where appropriate. Where appropriate, storage 606 may include one or more storages 606. Although this disclosure describes and illustrates particular storage, this disclosure contemplates any suitable storage.

In particular embodiments, I/O interface 608 includes hardware, software, or both, providing one or more interfaces for communication between computer system 600 and one or more I/O devices. Computer system 600 may include one or more of these I/O devices. where appropriate. One or more of these I/O devices may enable communication between a person and computer system 600. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 608 for them. Where appropriate, I/O interface 608 may include one or more device or software drivers enabling processor 602 to drive one or more of these I/O devices. I/O interface 608 may include one or more I/O interfaces 608, where appropriate. Although this disclosure describes and illustrates a particular I/O interface, this disclosure contemplates any suitable I/O interface.

In particular embodiments, communication interface 610 includes hardware, software, or both providing one or more interfaces for communication (such as, for example, packet-based communication) between computer system 600 and one or more other computer systems 600 or one or more networks. As an example and not by way of limitation, communication interface 610 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 610 for it. As an example and not by way of limitation, computer system 600 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 600 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 600 may include any suitable communication interface 610 for any of these networks, where appropriate. Communication interface 610 may include one or more communication interfaces 610, where appropriate. Although this disclosure describes and illustrates a particular communication interface, this disclosure contemplates any suitable communication interface.

In particular embodiments, bus 612 includes hardware, software, or both coupling components of computer system 600 to each other. As an example and not by way of limitation, bus 612 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 612 may include one or more buses 612, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

In particular embodiments, the specific kind of storage used for the data storage component of the system may be any of the many types of systems known in the art. These include, but are not limited to, systems such as traditional relational databases like PostgreSQL or MySQL, NoSQL systems like MONGODB or Berkeley DB, distributed systems like GOOGLE CLOUD SPANNER or AZURE COSMOS DB, or data lakehouse-based systems like DATABRICKS or SNOWFLAKE, among many others.

For this description herein, any reference to taking photos as they relate to the present system is intended to apply equally to recording a video. Given the current state of technology in photography and videography, it would be relatively easy for someone of ordinary skill in the relevant art to transfer the system design and functionality from photography to videography, or vice versa. In cases where that may not be the case, the description will provide explicit consideration on how the design and/or functionality may change when applied to photography and videography.

For this description herein, any mention of a specific feature relating to the system design or functionality is only intended as an example and not in any way as a limitation. Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

1. One or more computer-readable non-transitory storage media embodying software for guided self-capture of one or more images comprising code executable by a mobile computing device to:
display a graphical user interface (GUI) on a screen of the mobile computing device, wherein the GUI presents instructions to capture images of a specific area of the user's body posed in a predefined position, wherein the instructions comprise a graphical representation of the specific area of the user's body as posed in the predefined position;
upon detecting that a camera associated with the mobile computing device is enabled, display, in the GUI, images of a view appearing in a lens of the camera, wherein the graphical representation is displayed in conjunction with the images of the view;
capture one or more images of the view, wherein the captured images are stored on the mobile computing device;
display, in the GUI, the one or more stored images together with instructions to review the stored images; and
in response to receiving user input approving one or more of the stored images, transmit the approved one or more images over a network connection to a system associated with an image analyst.

2. The computer-readable non-transitory storage media of claim 1, wherein the instructions include at least one instruction describing how to manipulate the specific area of the user's body to achieve the predefined position, and wherein the graphical representation comprises a static image depicting the specific area of the user's body after it has been posed in the predefined position.

3. The computer-readable non-transitory storage media of claim 2, wherein the specific area of the user's body is an eye of the user, and wherein the at least one instruction describing how to manipulate the specific area of the eye comprises instructions to lift an eyelid of the eye using a finger, instructions to hold the eyelid in a raised position, and instructions to roll the eye downward and towards a nose of the user.

4. The computer-readable non-transitory storage media of claim 1, wherein the specific area of the user's body is an eye of the user, and wherein the predefined position is designed to reveal a device implanted within a specific area of a sclera of the eye.

5. The computer-readable non-transitory storage media of claim 4, wherein the graphical representation of the eye posed in the predefined position comprises a stylized image of an eye posed to reveal a large area of the sclera between an upper eyelid of the eye and an iris of the eye and a large area of the sclera between the iris and a lateral canthus of the eye, and wherein the upper eyelid is depicted as being raised high and the iris is depicted as being rolled downward and inward toward a medial canthus of the eye.

6. The computer-readable non-transitory storage media of claim 1, wherein the code is further executable to:
analyze the images of the view to assess compliance with a predefined standard for a usable diagnostic image of the specific area of concern, wherein the predefined standard for a usable diagnostic image comprises predefined standards for photographic properties of an image or a profile of a predefined position as depicted in a usable diagnostic image.

7. The computer-readable non-transitory storage media of claim 1, wherein the images of the view are analyzed as they appear in the lens of the camera;
upon determining that the view appearing in the lens of the camera satisfies the predefined standard for a usable diagnostic image of the specific area of concern, automatically cause the mobile device to activate the camera to capture the one or more images.

8. The computer-readable non-transitory storage media of claim 1, wherein the images of the view are analyzed after being stored, the code being further executable to;
determine that an unsatisfactory one of the stored images does not satisfy the predefined standard for a usable diagnostic image of the specific area of concern; and
display, in the GUI, the unsatisfactory image and an interactive element to capture a replacement image.

9. The computer-readable non-transitory storage media of claim 6, wherein the predefined standards for photographic properties of an image comprise brightness, sharpness, or contrast.

10. The computer-readable non-transitory storage media of claim 6, wherein the code executable to analyze the images of the view to assess compliance with a predefined standard for a usable diagnostic image of the specific area of concern comprises the code being executable to:
perform image analysis to identify one or more features of the specific area of the user's body; and
assess one or more measurable attributes of one or more of the identified features to determine compliance with the profile of the predefined position.

11. The computer-readable non-transitory storage media of claim 7, wherein the measurable attributes comprise: visibility of one of the identified features, a color of one of the identified features, an area of one of the identified features, a distance between two of the identified features, a proportion of one of the identified features to another one of the identified features, a shape of one of the identified features, a position of one of the identified features, a location of one of the identified features, an orientation of one of the identified features, a quantity of the identified features of a particular type, an angle associated with one or more identified features, or a ratio of measurable attributes.

12. The computer-readable non-transitory storage media of claim 6, wherein the profile of a predefined position as depicted in a usable diagnostic image comprises a minimum threshold, a maximum threshold, a ratio-based threshold, an acceptable range, an acceptable type, or an acceptable degree of deviation for the measurable attributes.

13. The computer-readable non-transitory storage media of claim 1, wherein the one or more stored images comprises a set of images capturing the view appearing in the lens of the camera over a short period of time in rapid succession.

14. The computer-readable non-transitory storage media of claim 1, wherein the code is further executable to:
display, in the GUI, an interactive element for taking a photo;
detect, by a touchscreen of the mobile computing device, a user interaction with the interactive element; and
in response to detection of the user interaction, cause the mobile computing device to store the one or more images.

15. The computer-readable non-transitory storage media of claim 1, wherein the code is further executable to:
detect, by a microphone of the mobile computing device, an audio command; and
in response to detection of the audio command, cause the mobile computing device to store the one or more images.

16. The computer-readable non-transitory storage media of claim 1, wherein the code is further executable to:
detect, by the camera of the mobile computing device, a gesture; and
in response to detection of the gesture, cause the mobile computing device to store the one or more images.

17. The computer-readable non-transitory storage media of claim 1, wherein each of the one or more images captured by the camera comprises a static image or a video.

18. The computer-readable non-transitory storage media of claim 15, wherein upon receiving an instruction that the one or more images should be captured by the camera, displaying a progress indicator during a period of time elapsing while the one or more images are being captured and stored.

19. A method comprising, by a mobile computing device:
displaying a graphical user interface (GUI) on a screen of the mobile computing device, wherein the GUI presents instructions to capture images of a specific area of the user's body posed in a predefined position, wherein the instructions comprise a graphical representation of the specific area of the user's body as posed in the predefined position;
upon detecting that a camera associated with the mobile computing device is enabled, displaying, in the GUI, a view appearing in a lens of the camera, wherein the graphical representation is displayed in conjunction with the view;
capturing one or more images of the view, wherein the captured images are stored on the mobile computing device;
displaying, in the GUI, the one or more stored images together with instructions to review the stored images; and
in response to receiving user input approving one or more of the stored images, transmitting the approved one or more images over a network connection to a system associated with an image analyst.

20. A system comprising one or more processors and a memory coupled to the processors comprising code executable by the processors, the processors being operable when executing the code to:
display a graphical user interface (GUI) on a screen of the mobile computing device, wherein the GUI presents instructions to capture images of a specific area of the user's body posed in a predefined position, wherein the instructions comprise a graphical representation of the specific area of the user's body as posed in the predefined position;
upon detecting that a camera associated with the mobile computing device is enabled, display, in the GUI, a view appearing in a lens of the camera, wherein the graphical representation is displayed in conjunction with the view;
capture one or more images of the view, wherein the captured images are stored on the mobile computing device;
display, in the GUI, the one or more stored images together with instructions to review the stored images; and
in response to receiving user input approving one or more of the stored images, transmit the approved one or more images over a network connection to a system associated with an image analyst.
